(19) United States
(12) Patent Application Publication
(10) Pub. No.: US 2021/0291142 A1
(43) Pub. Date: Sep. 23, 2021

(54) SINTERED BODY FOR ADSORPTION, PRODUCTION METHOD THEREFOR, AND ADSORPTION DEVICE

(71) Applicants: AJINOMOTO CO., INC., Tokyo (JP); NIPPON FILCON CO., LTD., Tokyo (JP)

(72) Inventors: Makoto Seino, Kanagawa (JP); Hideyuki Yano, Kanagawa (JP); Toshifumi Katoh, Tokyo (JP)

(73) Assignees: AJINOMOTO CO., INC., Tokyo (JP); NIPPON FILCON CO., LTD., Tokyo (JP)

(21) Appl. No.: 17/337,708

(22) Filed: Jun. 3, 2021

Related U.S. Application Data
(63) Continuation of application No. PCT/JP2019/047346, filed on Dec. 4, 2019.

(30) Foreign Application Priority Data
Dec. 5, 2018 (JP) 2018-227831

(51) Int. Cl. B01J 20/20; B01J 20/26; B01J 20/28; B01J 20/30; B01J 20/12; B01J 20/18; A61L 9/014; C02F 1/28; B01D 53/04

(52) U.S. Cl. CPC B01J 20/20; B01J 20/261; B01J 20/28004; B01J 20/28026; B01J 20/3078; B01J 20/12; C02F 2101/308; A61L 9/014; C02F 1/288; B01D 53/04; B01J 2220/46; C02F 1/283; C02F 1/285; B01J 20/18

(57) ABSTRACT

To adsorb a substance to be treated in a fluid (7) with a higher adsorption capacity and lower pressure loss, an adsorptive sintered compact (20) includes powder adsorbent materials (1a, 1b), and resin structures (2) in which voids (3) are formed in a three-dimensional network. The powder adsorbent materials (1a, 1b) include free adsorbent materials (1a) free-movably contained in the voids (3) between the resin structures (2), and fixed adsorbent materials (1b) fixed to a surface (2a) of the resin structure (2) and/or at least partly embedded inside the resin structure (2), and the powder adsorbent materials (1a, 1b) are at least one of powdered activated carbon, powdered activated clay, and zeolite.

This application is a Continuation of, and claims priority under 35 U.S.C. § 120 to, International App. No. PCT/JP2019/047346, filed 4 Dec. 2019, and claims priority therethrough to Japanese App. No. 2018-227831, filed 5 Dec. 2018, the entireties of which are incorporated by reference herein.

### BACKGROUND

### Field of Endeavor

The present disclosure relates to a sintered object for adsorption, and more particularly to an adsorptive sintered compact which contains powder adsorbent materials for adsorbing substance(s) to be treated in fluid, a production method therefor, and an adsorption apparatus.

### Brief Description of the Related Art

Adsorbent materials such as activated carbon, activated clay, and zeolite, etc., are used for various adsorption uses for industrial, household, and medical purposes including air purification, dioxin removal, flue gas desulfurization and denitrification, odor removal, waste gas and liquid treatment of a factory, advanced water purification, purification of chemicals, decolorization of foods and beverages, water purifiers for home use, air purifiers, refrigerator deodorants, gas masks, etc.

For example, Patent Document 1 discloses an activated carbon cartridge for gas purification in which granular activated carbon having a particle diameter of 2.4 to 4.7 mm (2,400 to 4,700 μm) is filled between an inner cylinder and an outer cylinder. In general, “granular” activated carbon refers to activated carbon having a larger particle diameter, and fine powder activated carbon having smaller particle diameter is called “powdered” activated carbon. In JIS K 1474, a particle diameter indication of 150 μm or more is defined as “granular” activated carbon and less than 150 μm is “powdered” activated carbon. Granular adsorbent material having a large particle diameter has a small specific surface area (outer surface area per unit mass) compared with that of a fine powder adsorbent material, so that in order to obtain the same level of adsorption performance as when a powder adsorbent material is used, it is necessary to increase the amount of granular adsorbent material, thus when a granular adsorbent material is used as a filter by filling in a cartridge of an adsorption apparatus, the cartridge or adsorption apparatus becomes large. Also, when a granular adsorbent material is tried to be filled tightly in a vessel in order to heighten the adsorption performance, the filling volume is likely to vary from vessel to vessel as compared with that of a powder adsorbent material, so that there may be a problem that it is not easy to maintain constant quality in the manufacture of a product such as a cartridge for a filter. In addition, it is sometimes inconvenient that pressure loss increases since when a granular adsorbent material in a vessel is pulverized, destroyed, or the like, due to external forces such as vibration and pressure at the time of transportation or use.

On the other hand, a powder adsorbent material, such as powdered activated carbon, has a larger specific surface area and higher adsorption performance as compared with that of a granular adsorbent material, but even if it is used as a filter by filling it in a vessel such as a cartridge, voids between the powder adsorbent materials are extremely reduced and fluid passes through with difficulty, and even if it passes through, it causes a high pressure loss (high differential pressure) and thus it is not suitable for practical use. For this reason, powder adsorbent material is not suitable for a filter by filling in a vessel, such as a cartridge, and therefore has been conventionally used exclusively for a batch type adsorption process.

In an attempt to improve the pressure loss in a vessel filled with adsorbent material, for example, Patent Document 2 discloses an adsorptive molded product based on an agglomerate formed by binding and/or adhering adsorbent material through a binder of thermoplastic resin, and discloses a filter to which it is applied. However, in such an agglomerate, most of the surface of adsorbent material is covered by thermoplastic resin, so that adsorption performance is lowered. Particularly, for a powder adsorbent material having a small particle diameter, most of its surface tends to be covered by thermoplastic resin, and it is difficult to make an agglomerate into a desired size due to a decrease in pressure loss, so that in the Examples section of Patent Document 2, granular activated carbon having a large particle diameter is used practically.

Further, in an attempt to resolve the problem that the surface of an adsorbent material contained in an adsorptive molded product is covered by thermoplastic resin, for example, Patent Document 3 discloses a molded product in which a foaming agent is impregnated or adsorbed to highly functional particles such as activated carbon particles and mixed with matrix resin, and the mixture is solidified by foaming in a liquid or molten state of a matrix resin whereby highly functional particles are allowed to exist inside pores generated by the foaming.

However, when such a so-called foamed plastic is used as a matrix of adsorbent material, the molded product is generally flexible and poor in mechanical strength, and particularly is easily deformed under high pressure and high flow rate, so that it is not suitable for a continuous system adsorption apparatus which adsorbs substance to be treated in a fluid. Specifically, for example, as shown in FIG. 14(a) and FIG. 15(a), as compared with molded product 10 before use, molded product 10 at the time of use under high pressure or high flow rate is easily crushed in its entire volume by compressive force 16 in FIG. 14(b) since it is constituted by foamed plastic 12, and as shown in FIG. 15(b), pores 13 tend to shrink as compared with those before use so that the density of activated carbon particles 11 becomes high and thus pressure loss increases and the flow rate markedly lowers.

In addition, since pores are formed in a matrix resin by impregnating a foaming agent into an adsorbent material, if the foaming agent is not sufficiently impregnated into the adsorbent material, it is not easy to form suitable pores therein. Furthermore, when the content of adsorbent material is increased for the purpose of increasing adsorption performance, pores also increase and mechanical strength tends to lower, so that it is difficult to improve the adsorption efficiency.

Also, an open cell structure which is said to be preferable in an obtainable molded product becomes a so-called sponge-like structure, and further mechanical strength is reduced, so that there is a problem that it is not suitable for the use of a continuous treatment for adsorbing substances to be treated in a fluid (7) as used by filling it in vessel such as a cartridge as mentioned above.

### PRIOR ART DOCUMENTS

### Patent Documents

Patent Document 1: JP2014-104448A
Patent Document 2: JP2012-508645A
Patent Document 3: JPH01-301732A

### SUMMARY

Thus, an object of the present disclosure is to provide an adsorptive material which is excellent in adsorption capacity and capable of achieving lower pressure loss simultaneously, and a production method therefor, and an adsorption apparatus.

The present inventors have earnestly studied to accomplish the above-mentioned object, and as a result, they have found that, a specific adsorptive sintered compact comprising: resin structure(s) in which voids are formed to a three-dimensional network; and powder adsorbent materials contained in voids free-movably and simultaneously fixed to a surface of resin structure(s) and/or at least a part thereof embedded inside resin structure(s) Such a compact may have excellent adsorption capacity and be simultaneously capable of achieving lower pressure loss.

That is, the present disclosure includes the following.

An adsorptive sintered compact which comprises:

powder adsorbent materials (1a, 1b); and

a resin structure (2) in which voids (3) are formed in a three-dimensional network;

wherein the powder adsorbent materials (1a, 1b) include:

a free adsorbent material (1a) free-movably contained in the voids (3) between the resin structures (2); and

a fixed adsorbent material (1b) fixed to a surface (2a) of the resin structure (2) and/or at least partly embedded inside the resin structure (2); and

the powder adsorbent materials (1a, 1b) are at least one selected from powdered activated carbon, powdered activated clay, and zeolite.

The adsorptive sintered compact described above, wherein a mean diameter of the powder adsorbent materials (1a, 1b) is less than 150 μm.

The adsorptive sintered compact described above, wherein the powder adsorbent materials (1a, 1b) are contained in an amount of 25 to 65 weight %.

The adsorptive sintered compact described above, wherein a resin raw material of the resin structure (2) is at least one thermoplastic resin selected from polypropylene (PP), polyethylene (PE), polyvinylidene fluoride (PVDF), and ethylene-vinyl acetate (EVA) copolymer.

The adsorptive sintered compact described above, wherein a particle diameter of the thermoplastic resin is 10 to 200 μm.

The adsorptive sintered compact described above, wherein a plurality of free adsorbent materials (1a) are free-movably contained in at least a part of the voids (3), and the plurality of the adjacent free adsorbent materials (1a) form a flow path (3a) of a fluid (7) between free adsorbent materials (1a) in at least a part of voids (3) without fixation of the adjacent free adsorbent materials to each other.

The adsorptive sintered compact described above, wherein the powder adsorbent material (1a, 1b) is powdered activated carbon having a sharp portion.

The adsorptive sintered compact described above, which is used for adsorbing a substance to be treated in the fluid (7).

An adsorption apparatus which comprises a single or a plurality of layers (20a, 20b) of the adsorptive sintered compact (20) described aboveloaded in a vessel.

A method for producing an adsorptive sintered compact which comprises:

mixing a powder adsorbent material which is at least one selected from powdered activated carbon, powdered activated clay, and zeolite, with a thermoplastic resin to form an adsorbent material mixture;

heating the adsorbent material mixture at a temperature higher than a softening point of the thermoplastic resin and lower than a melting point of a raw material of the powder adsorbent material; and

forming a resin structure (2) in which a plurality of the thermoplastic resins are fused and solidified by cooling to form voids (3) in a three-dimensional network and a free adsorbent material (1a) is free-movably contained in the void (3).

The production method described above, wherein a mean diameter of the powder adsorbent material is less than 150 μm.

The production method described above, wherein a content of the powder adsorbent material is 25 to 65 weight % in the adsorbent material mixture.

The production method described above, wherein the thermoplastic resin is at least one selected from polypropylene (PP), polyethylene (PE), polyvinylidene fluoride (PVDF), and ethylene-vinyl acetate (EVA) copolymer.

The production method described above, wherein a particle diameter of the thermoplastic resin is 10 to 200 μm.

According to the present disclosure, it is possible to provide an adsorptive material which is excellent in adsorption capacity and capable of simultaneously achieving lower pressure loss, and a production method therefor, and an adsorption apparatus.

### BRIEF DESCRIPTION OF THE DRAWINGS

FIG. 1 is a partial cross-sectional view showing an adsorptive sintered compact of the present disclosure.

FIG. 2a is an enlarged surface image showing powdered activated carbon as a raw material.

FIG. 2b is an enlarged surface image showing powdered activated carbon as raw material.

FIG. 2c is an enlarged surface image showing powdered activated carbon as raw material.

FIG. 2d is an enlarged surface image showing powdered activated clay as raw material.

FIG. 2e is an enlarged surface image showing zeolite as a raw material.

FIG. 3 is an enlarged cross-sectional image showing a resin structure alone of an adsorptive sintered compact.

FIG. 4a is a surface image showing an adsorptive sintered compact of the present disclosure produced from powdered activated carbon.

FIG. 4b is an enlarged surface image showing an adsorptive sintered compact of the present disclosure produced from powdered activated carbon.

FIG. 4c is an enlarged surface image showing an adsorptive sintered compact of the present disclosure produced from powdered activated carbon.

FIG. 4d is an enlarged surface image showing an adsorptive sintered compact of the present disclosure produced from powdered activated clay.

FIG. 4e is an enlarged surface image showing an adsorptive sintered compact of the present disclosure produced from zeolite.

FIG. 5 is a cross-sectional view showing an adsorption apparatus loaded with an adsorptive sintered compact of the present disclosure.

FIG. 6 is a partial cross-sectional view showing an embodiment in which liquid or gas is passed through an adsorptive sintered compact of the present disclosure.

FIG. 7 is a schematic view showing a pressure loss test apparatus.

FIG. 8 is a graph showing results of a pressure loss test.

FIG. 9 is a graph showing results of a pressure loss test.

FIG. 10 is a schematic view showing a liquid flow adsorption test apparatus.

FIG. 11a is a graph showing results of a liquid flow adsorption test using coconut shell activated carbon.

FIG. 11b is a graph showing results of a liquid flow adsorption test using sawdust activated carbon.

FIG. 12 is a schematic view showing a gas adsorption test apparatus.

FIG. 13 is a graph showing results of a gas adsorption test.

FIG. 14 is a cross-sectional view showing a state in which a conventional molded product is loaded in vessel.

FIG. 15 is a partial cross-sectional view showing a conventional molded product.

### DETAILED DESCRIPTION OF EXEMPLARY EMBODIMENTS

An adsorptive sintered compact of the present disclosure is provided with powder adsorbent materials (1a, 1b), and resin structure(s) (2) in which voids (3) are formed as a three-dimensional network shape. The resin structure (2) in the present disclosure can be formed by heating particles of a thermoplastic resin, such as powder, granules, pellets, and melting contacted portions of a plurality of thermoplastic resins to form joint portions, whereby the thermoplastic resins are thermally bonded to each other. The resin structures thus obtained have structures in which concave voids sandwiched between convex portions derived from the shape of the thermoplastic resin are formed in a three-dimensional network. The powder adsorbent materials (1a, 1b) contain a free adsorbent material (1a) free-movably contained in voids (3) between the resin structures (2), and a fixed adsorbent material (1b) fixed to the surface (2a) of the resin structure (2) and/or at least partly embedded inside the resin structure (2). The powder adsorbent materials (1a, 1b) are constituted by at least one of powdered activated carbon, powdered activated clay, and zeolite.

An adsorptive sintered compact of the present disclosure is useful particularly for adsorbing substances to be treated in a fluid (7). When the fluid (7) is passed through the adsorptive sintered compact (20), the entire surface of the free adsorbent material (1a) which is not fixed to the resin structure (2) and not joined, comes into direct contact with the fluid (7), so that it can capture and adsorb the substances to be treated in the fluid (7). In addition, in the present disclosure, not only the free adsorbent material (1a) of the voids (3), but also the fixed adsorbent material (1b) is provided in the resin structure (2), so that the adsorptive sintered compact (20) of the present disclosure can ensure a larger adsorption area as compared with conventional adsorbent materials including the type in which the entire surface or a part of surface is coated by matrix resin and the type in which the adsorbent materials exist only in voids, and thus the adsorption efficiency is markedly increased, and improvement in the adsorption performance can be accomplished.

Furthermore, when the fluid (7) is passed through the adsorptive sintered compact (20), due to voids (3) being formed in the three-dimensional network shape, the free adsorbent materials (1a) are freely movable while the movement thereof is restricted with a certain extent and the agglomeration thereof is suppressed. Therefore, it is possible to suppress clogging of voids (3) and gaps (4) between the resin structures (2), and as a result, even when powder adsorbent materials (1a, 1b) are used, the increase in pressure loss in an adsorptive sintered compact (20) is suppressed, and continuously adsorbing substances to be adsorbed in the fluid (7) can occur for a long period of time.

Moreover, the substances to be adsorbed and captured by the free adsorbent material (1a) can be removed from the flowing and free-moving adsorbent materials (1a) if the conditions are selected, and the adsorptive sintered compact (20) can be reused. That is, for removing the adsorbed substances, for example, a method can be selected in which the adsorptive sintered compact (20) is heated within a structurally maintainable range, and extracted by adding a good solvent of the adsorbed substance. In addition, it is also possible to select a method in which removal is facilitated by reducing a pressure when it is a gas phase or by changing liquid properties such as pH and a concentration of salt when it is a liquid phase. Furthermore, a plurality of these methods capable of being used in parallel can be used in combination. In the adsorptive sintered compact of the present disclosure, in order to enhance the removal ability of the substance to be adsorbed, it is possible to carry metals such as silver, copper, nickel, and metal oxides thereof, and/or non-volatile chemicals such as acids and/or bases, on the raw material of the powder adsorbent material in advance. Further, these can be directly carried on the adsorptive sintered compact.

In the adsorption apparatus of the present disclosure, a single or a plurality of layers (20a, 20b) of the adsorptive sintered compact (20) is loaded in a vessel. As compared with the case where the powdered activated carbon is directly filled in the vessel, the adsorption apparatus (30) is excellent in strength characteristics due to the rigid resin structure (2), and a certain form and voids (3) can be maintained at the time of transportation and use. Further, even if a plurality of layers (20a, 20b) of the adsorptive sintered compact (20) are loaded, the respective layers (20a, 20b) do not mix.

A method for producing the adsorptive sintered compact of the present disclosure comprises steps of: mixing at least one powder adsorbent raw material selected from powdered activated carbon, powdered activated clay, and zeolite, with a thermoplastic resin to form an adsorbent material mixture; heating the adsorbent material mixture at a temperature higher than the softening point of the thermoplastic resin and lower than the melting point of the powder adsorbent raw material; and fusing a plurality of the thermoplastic resins and solidifying by cooling to form a resin structure (2).

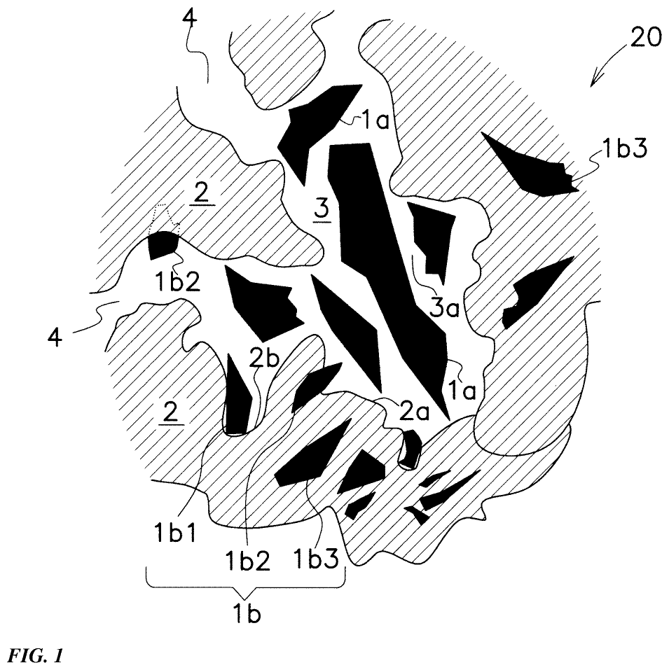

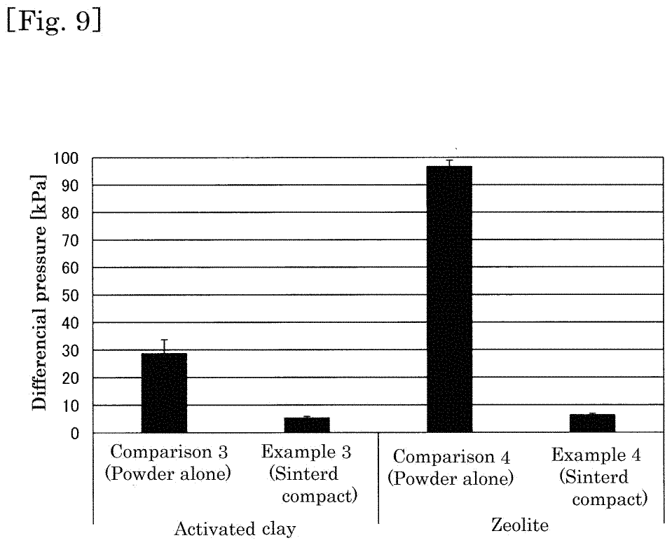

Embodiments of an adsorptive sintered compact and a production method therefor according to the present disclosure will be explained in more detail below by referring to FIG. 1 to FIG. 13.

An exemplary embodiment of an adsorptive sintered compact (20) of the present disclosure is shown in FIG. 1. Adsorptive sintered compact (20) is provided with powder adsorbent materials (powder adsorbent agents) (1a, 1b) that adsorb substance(s) to be treated in a fluid (7), and resin structure(s) (2) in which voids (cavities) (3) are formed in a three-dimensional network. The substance to be treated is contained in a gas or liquid, and may include all components and substances adsorbable to powder adsorbent materials (1a, 1b), for example, a color component, odor component, a harmful substance, a pollutant, a heavy metal, a valuable metal, a toxic component, a radioactive component, water, oil, etc.

Powder adsorbent materials (1a, 1b) in the present disclosure are at least one of powdered activated carbon, powdered activated clay, and zeolite. Adsorptive sintered compact (20) may contain other adsorbent substance(s), for example, acid clay, alumina, silica, silica gel, silica-alumina, vermiculite, perlite, kaolin, diatomaceous earth, sepiolite, etc., to the extent that effects of the present disclosure can be achieved. Also, in order to further decrease pressure loss when the adsorptive sintered compact is used for an adsorption apparatus, adsorptive sintered compact (20) may contain granular adsorbent materials (adsorbent materials having mean diameter of 150 μm or more) such as granular activated carbon, within a range in which the adsorption capacity is not extremely reduced. Activated carbon used for the powder adsorbent materials (1a, 1b) is formed by activating a raw material, for example, coconut shell, walnut shell, apricot shell, fruit shell, paddy shell, soybean, coffee, nuts, pistachio, charcoal, sawdust, bark, carbon from sawdust, wood material, peat, grass peat, lignite, brown coal, bituminous coal, anthracite, tar, pitch, coke, coal, petroleum, waste tires, waste plastic, synthetic resin, fiber, construction waste, and sewage sludge, and the adsorption performance is imparted by innumerable internal micropores. As an activation method, for example, a gas activation with water vapor, carbon dioxide, air, etc., and a chemical activation with zinc chloride, phosphoric acid, sulfuric acid, calcium chloride, potassium dichromate, potassium permanganate, sodium hydroxide, etc., can be applied.

A mean diameter of powder adsorbent material in the present disclosure is not particularly limited as long as the effect of the present disclosure can be achieved, but it is preferably less than 150 μm. That is, in the present specification, adsorbent material preferably having a mean diameter of less than 150 μm is defined to be a powder adsorbent material. Also, “powder” adsorbent material having a particle diameter indication of less than 150 μm according to JIS K 1474 is also included in the powder adsorbent material of the present disclosure. “Mean diameter” of the present specification is measured by the laser diffraction scattering method based on the Mie scattering theory. Specifically, particle size distribution of powder adsorbent raw material is prepared based on a volume basis by the laser diffraction scattering particle analyzer (LA-500 manufactured by HORIBA, Ltd., LMS-2000e manufactured by SEISHIN ENTERPRISE Co., Ltd.), and the median diameter is taken as the mean diameter. Incidentally, for example, a mean diameter of adsorbent material of less than 150 μm includes any adsorbent materials having median diameter of less than 150 μm. That is, the value of mean diameter merely represents the intermediate value of distribution range, and for example, the mean diameter of adsorbent material less than 150 μm does not mean that adsorbent material having diameter of 150 μm or more is not included at all. Mean diameter of powder adsorbent material may be 1 μm or more to less than 150 μm, may be 5 μm or more to less than 150 μm, and may be 15 μm or more to 100 μm or less. The mean diameter of the powder adsorbent materials (1a, 1b) contained in the adsorptive sintered compact (20) can be adjusted by setting the mean diameter of powder adsorbent material to be used at the time of producing the adsorptive sintered compact (20) within the above numerical range. That is, the mean diameter of the powder adsorbent material to be used at production can become the mean diameter of the powder adsorbent materials (1a, 1b) contained in the adsorptive sintered compact (20).

If the mean diameter of the powder adsorbent materials (1a, 1b) is too small, a large amount of fine powder adsorbent materials (1a, 1b) is mixed with resin raw materials of resin structure (2) at the production, so that there is a possibility of reducing the strength of the resin structure (2). Also, there is a possibility that fine free adsorbent material (1a) may easily drop out from the adsorptive sintered compact (20) with fluid (7) through gap (4) between resin structures (2). Also, there is a possibility that pressure loss is easily increased. If the mean diameter is too large (for example, if it is 150 μm or more), the adsorption area of the adsorbent material is small and the adsorption performance tends to be reduced. Also, a sufficient amount of free adsorbent material (1a) cannot be contained in voids (3) and, therefore, the constitution of the adsorptive sintered compact (20) tends to be difficult.

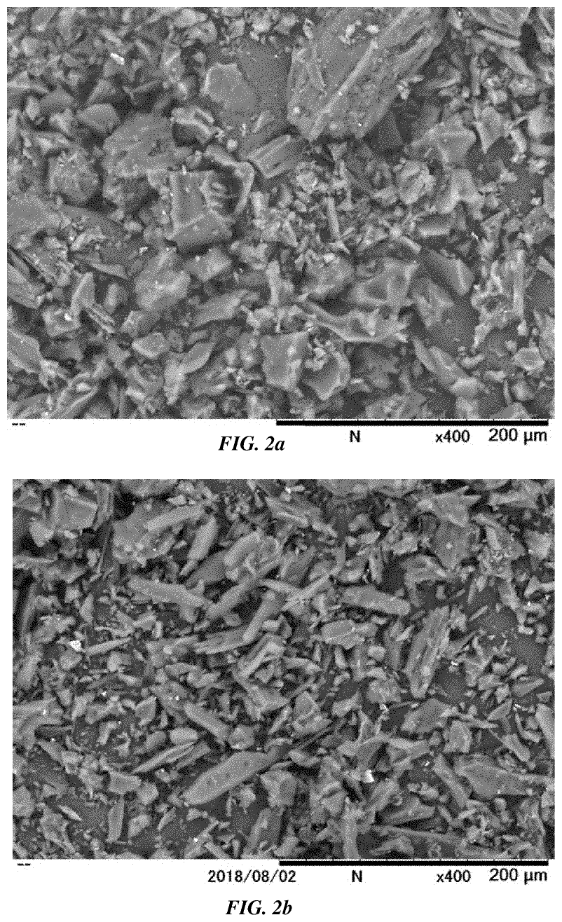

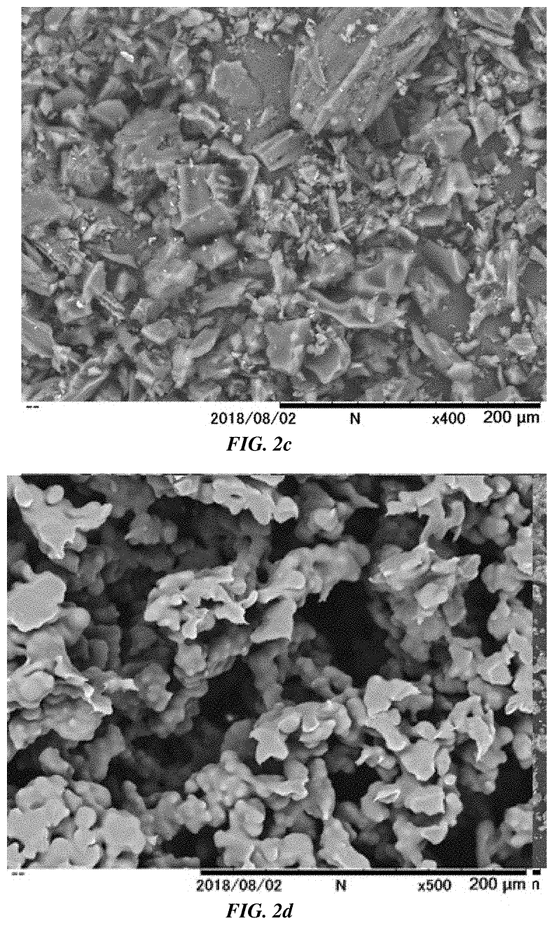

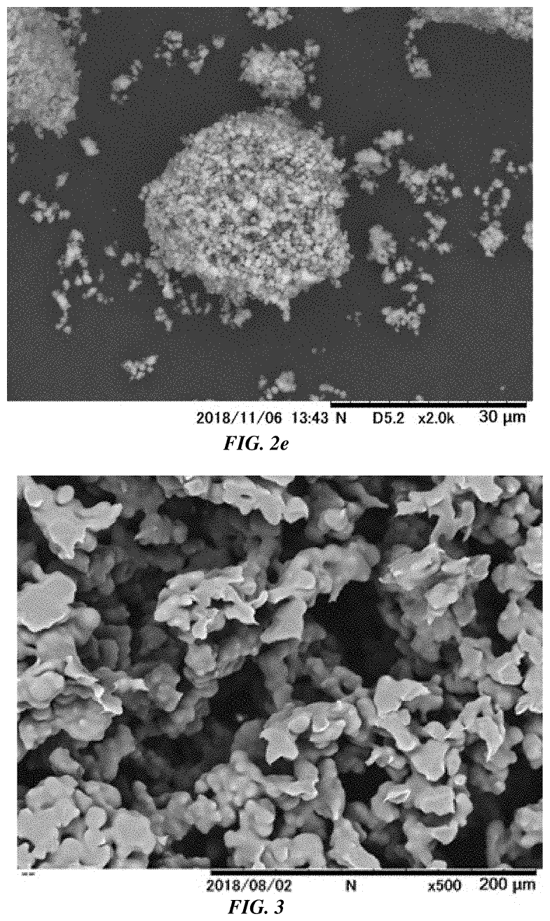

FIG. 2a to FIG. 2e are electron micrograph images showing raw materials of powder adsorbent materials (1a, 1b) for constituting the adsorptive sintered compact (20). In particular, FIGS. 2a, 2b, and 2c show enlarged surface images (400-fold) of powdered activated carbons obtained by respectively activating: carbide of coconut shell with water vapor; sawdust with chemicals (phosphoric acid); and sawdust with chemicals (zinc chloride). Powdered activated carbon is generally a lumpy, rod-shaped or elongated plate-shaped raw material having sharp portion(s), so that a tip of the powder adsorbent materials (1a, 1b) locks in surface (2a) and gap (4) of resin structures (2) when the powder adsorbent materials are freely moving, to prevent from flowing out of the adsorptive sintered compact (20). Therefore, the powder adsorbent materials (1a, 1b) hardly move from void to void (3). FIG. 2d and FIG. 2e are enlarged surface images each showing powdered activated clay (400-fold) and zeolite (2,000-fold) as the powder adsorbent raw material. An adsorptive sintered compact (20) may contain powder adsorbent materials (1a, 1b) of 25 to 65 weight %, and may contain 30 to 60 weight %. If the content of powder adsorbent materials (1a, 1b) is too low, the adsorption performance of the adsorptive sintered compact (20) tends to be reduced. On the other hand, if the content of the powder adsorbent materials (1a, 1b) is too high, it has a high adsorption capacity but the ratio of resin is small and the strength of entire adsorptive sintered compact (20) tends to be reduced. In the present disclosure, the content of the powder adsorbent materials (1a, 1b) contained in an adsorptive sintered compact (20) can be defined as that of a powder adsorbent material contained in an adsorbent material mixture before sintering to be used in the production of a compact, and therefore can be adjusted by appropriately setting the content of powder adsorbent material in the adsorbent material mixture.

A thermoplastic resin is used as a resin raw material of the resin structure (2). It is preferably at least one thermoplastic resin from among polypropylene (PP), polyethylene (PE), polyvinylidene fluoride (PVDF), and ethylene-vinyl acetate (EVA) copolymer. A thermoplastic resin as the resin raw material of the resin structure (2) is used in a particulate solid, and may be in any form such as powder, granules, and/or pellets. The particle diameter of the thermoplastic resin particle is preferably 10 to 200 μm. By setting the particle diameter of the thermoplastic resin within the above-mentioned range, it becomes easy to form the size of voids (3) into substantially uniform dimensions without variation. Plasticizer such as adipic acid ester, stabilizer such as epoxy-based compound, and antioxidant such as phenol-based compound can be added to the thermoplastic resin depending on the kind and characteristics thereof.

The particle diameter of the thermoplastic resin in the present disclosure is measured by an image analysis method photographed by a CCD camera. Specifically, 1,400 to 15,000 randomly dispersed particles of thermoplastic resin are photographed by an image analysis type particle distribution measuring device (VD-3000 manufactured by JASCO International Co., Ltd., PITA-04 manufactured by SEISHIN ENTERPRISE Co., Ltd., etc.) to obtain images which calculate the individual particle diameter D of each particle, and after preparing the distribution of D, the median diameter is made to be the particle diameter of thermoplastic resin. Incidentally, an individual particle diameter D can be obtained from a maximum diameter D1 of maximum width and a minimum diameter D2 of minimum width of a particle in the image and from the arithmetic mean (D1+D2)/2.

FIG. 3 is a cross-sectional enlarged image (500×) by an electron microscope of an adsorptive sintered compact containing no powder adsorbent materials (1a, 1b), and shows the state of cross-section in which resin raw material alone is sintered to form resin structure and cut by a cutter. The white portion of FIG. 3 shows resin structures (2) of a continuous tufted and three-dimensional network structure without any corners, and the black part inside thereof shows voids (3) in which free adsorbent materials (1a) are to be freely movably arranged.

The powder adsorbent materials (1a, 1b) of the adsorptive sintered compact (20) shown in FIG. 1 contain free adsorbent materials (free adsorbent agents) (1a) free-movably contained in voids (3) between resin structures (2). The term “free-movably” herein means that a powder adsorbent material is not fixed to a resin structure (2) or a plurality of powder adsorbent materials are not fixed to each other, and that all kinds of movement such as shifting, rocking, vibration, rotation, extension/shrinkage, expansion/contraction, floating/sinking, etc., are possible.

The powder adsorbent materials (1a, 1b) of the adsorptive sintered compact (20) shown in FIG. 1 may also be provided with fixed adsorbent materials (1b) carried on the resin structure (2). Fixed adsorbent materials (1b) are firmly fixed to the surface (2a) of the resin structure (2), or at least partly embedded inside the resin structure (2). More specifically, a fixed adsorbent material (1b) may include: a first fixed adsorbent material (1b1) fixed to a surface (2a) of a resin structure (2); a second fixed adsorbent material (1b2) in which a part thereof (a part of its surface) is embedded inside a resin structure (2) and the remainder (the remainder of its surface) protrudes from the resin structure (2); and/or a third fixed adsorbent material (1b3) in which its entirety (its entire surface) is embedded inside a resin structure (2). In fixed adsorbent materials (1b), the first and second fixed adsorbent materials (1b1, 1b2) in which surfaces thereof are exposed to voids (3) contribute to improvement in adsorption ability, and do not flow outside because they are fixed to the resin structure (2) even they are small in diameter. Although the third fixed adsorbent material (1b3) is included in resin structure (2) and has low adsorption ability, the powder adsorbent materials (1a, 1b) having a smaller diameter tend to be easily incorporated into resin structure (2), and the fixed adsorbent material (1b3) is constituted by the powder adsorbent material having a relatively small diameter, and thus it can prevent clogging caused by a large amount of powder adsorbent materials (1a, 1b) having a smaller diameter remaining in voids (3) and can thus suppress an increase in pressure loss.

In production steps of an adsorptive sintered compact (20), the smaller the particle diameter of the powder adsorbent materials (1a, 1b) is, the more fixed they are to the surface (2a) of the resin structure (2) or at least a part thereof and tend to be embedded inside resin structure (2), so that the fixed adsorbent material (1b) tends to have a relatively smaller particle diameter as compared with that of the free adsorbent material (1a). On the other hand, the free adsorbent material (1a) tends to have a relatively large particle diameter. Therefore, when a part or all of the free adsorbent material is recovered by cutting an adsorptive sintered compact to measure the mean diameter thereof, the mean diameter of free adsorbent material is generally larger than the mean diameter of the powder adsorbent material, as compared with that of the powder adsorbent material used as a raw material of the adsorptive sintered compact (20). The mean diameter of the free adsorbent material (1a) may be 5 μm or more and less than 150 μm, and may be 15 μm or more and 100 μm or less. The mean diameter of the fixed adsorbent material (1b) is estimated to be 1 to 50 μm or so.

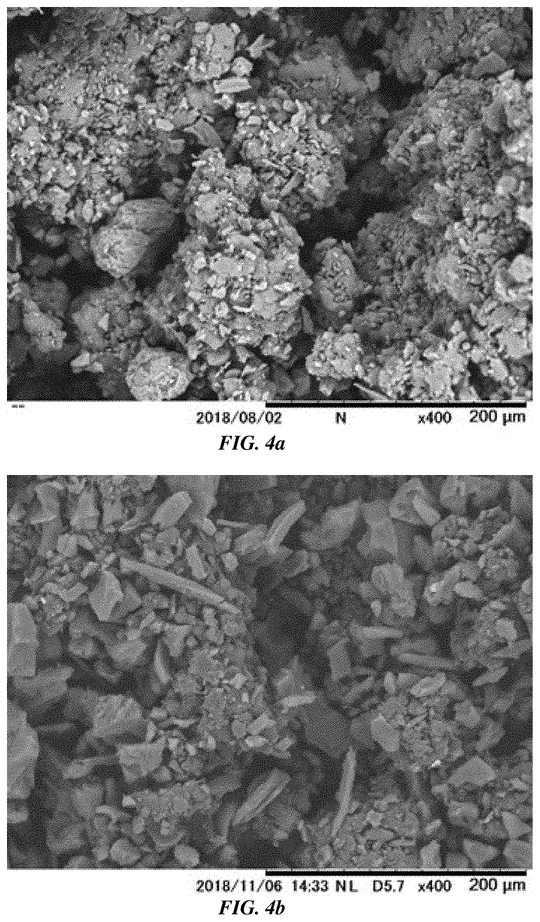

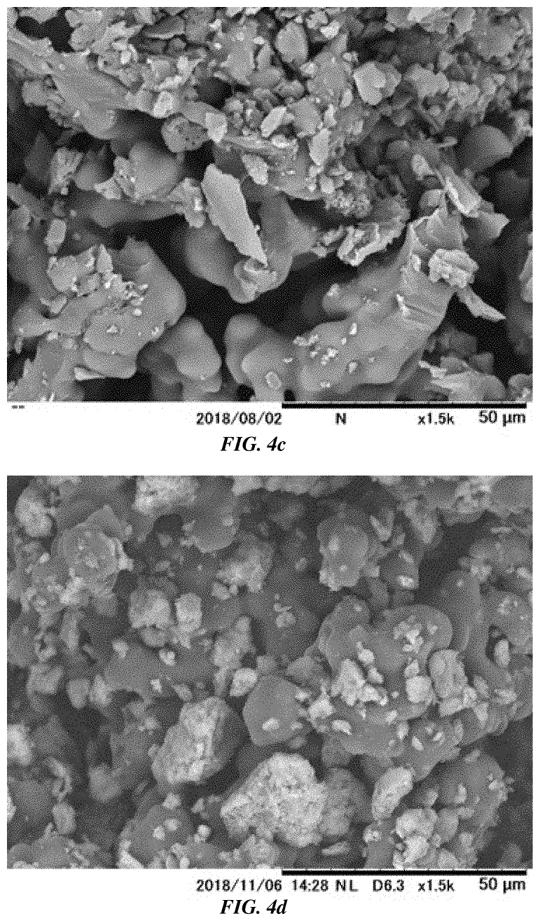

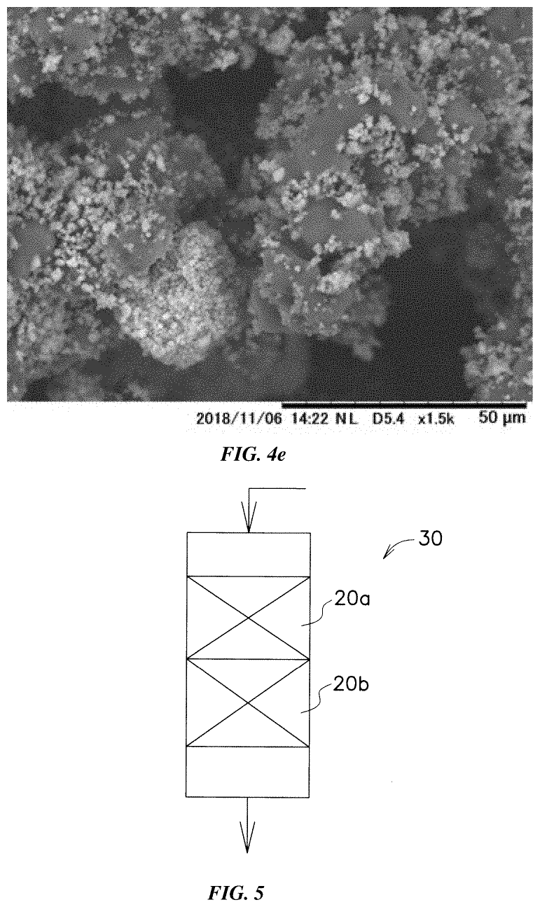

FIG. 4a to FIG. 4c show cross-sectional enlarged surface images of adsorptive sintered compacts (20) of the present disclosure made of, as powder adsorbent raw materials: powdered activated carbon (FIG. 2a) obtained by activating carbide of coconut shell with water vapor; powdered activated carbon (FIG. 2b) obtained by activating sawdust with a chemical (phosphoric acid); and powdered activated carbon (FIG. 2c) obtained by activating sawdust with a chemical (zinc chloride); which were respectively photographed by an electron microscope of each magnification. In each case, the images confirm adsorptive sintered compact (20) having: resin structures (2) having smooth rounded surfaces (2a) with black-colored voids (3) formed into a three-dimensional network shape; free adsorbent materials (1a) of powdered activated carbon provided inside the resin structures (2); and fixed adsorbent materials (1b) of powdered activated carbon fixed to the resin structures (2). Black voids (3) inside the resin skeleton constitute a three-dimensional space in which free adsorbent materials (1a) can freely move. A part of the fixed adsorbent materials (1b) is fixed to the surface (2a) of the resin structure (2), and a part thereof is fitted to a concave portion (2b) of the surface (2a) and can be held in the resin structure (2).

In the present disclosure, not only free adsorbent materials (1a) in voids (3), but also fixed adsorbent materials (1b) are provided in resin structures (2), so that a higher adsorption performance can be maintained by increasing the adsorption surface and the saturated adsorption capacity. In other words, the resin structure (2) alone has no adsorption ability unless a specific functional substance is added, so that the adsorption performance of the entire adsorptive sintered compact (20) can be significantly improved by carrying fixed adsorbent materials (1b) on the surfaces (2a) or inside of the resin structures (2). Also, in the present disclosure, fixed adsorbent materials (1b) do not flow out from the gap (4) between the resin structures (2).

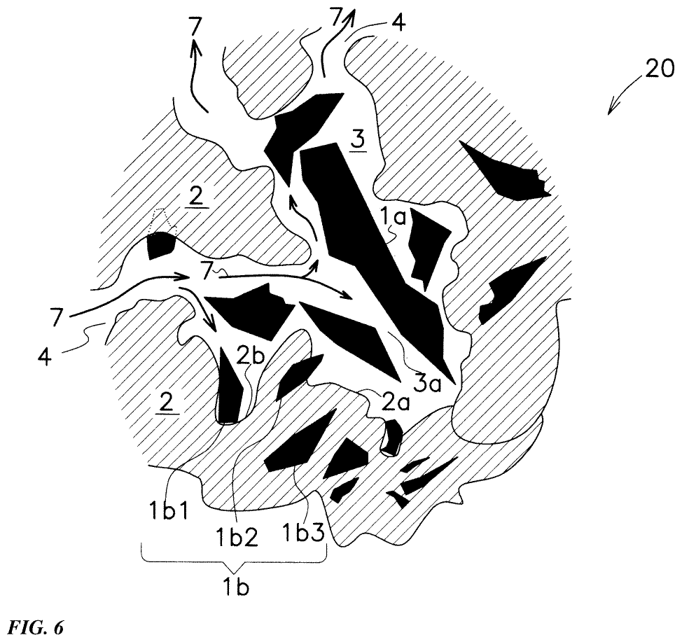

As can be confirmed from FIG. 4a to FIG. 4c, one or more free adsorbent material(s) (1a) is/are free-movably contained in voids (3). When two or more free adsorbent materials (1a) are contained in one void (3), these free adsorbent materials (1a) adjacent to each other form a flow path (3a) (FIG. 1 and FIG. 6) for fluid (7) between the free adsorbent materials (1a) of at least a part of voids (3) without fixing to each other to enable the full surface adsorption of the free adsorbent materials (1a). Even if a free adsorbent material (1a) once contacts with another free adsorbent material (1a) or resin structure (2), it can separate due to the flow of fluid (7) and form a flow path (3a) again.

FIG. 4d and FIG. 4e show cross-sectional enlarged surface images (1,500×) photographed by an electron microscope that show that adsorptive sintered compacts (20) of the present disclosure are respectively made of, as raw material of powder adsorbent material, activated clay (FIG. 2d) and zeolite (FIG. 2e). From FIG. 4d and FIG. 4e, adsorptive sintered compacts (20) can be seen having: grey resin structures (2) having a smooth rounded surface (2a) in which black-colored voids (3) are formed in a three-dimensional network shape; large-diameter powdered activated clay and zeolite as free adsorbent material (1a) provided inside the resin structures (2); and small-diameter powdered activated clay and zeolite as a fixed adsorbent material (1b) fixed to the resin structures (2).

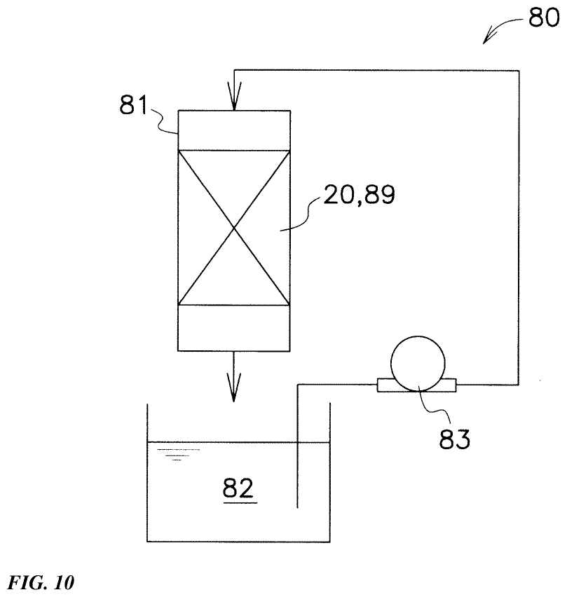

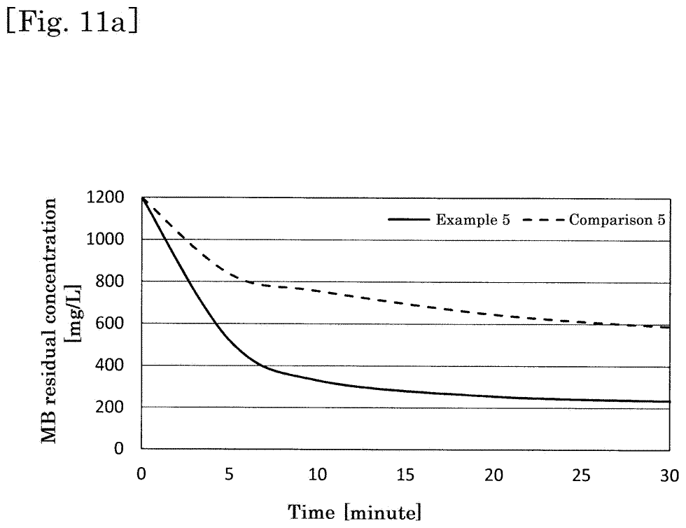

An adsorption apparatus of the present disclosure, in which an adsorptive sintered compact (20) is filled, may be formed by stacking a single layer or a plurality of layers of an adsorptive sintered compact (20), for example, into a vessel as shown by reference numeral (81) in FIG. 10. As compared with the conventional technology in which granular adsorbent materials are directly filled in a vessel, an adsorption apparatus of the present disclosure can maintain a certain form and voids (3) due to resin structures (2) having high mechanical strength without causing problems, such as pulverization and breakage of the adsorbent materials in the vessel and an increase in pressure loss, at the time of transportation and use. FIG. 5 shows a two-layer (20a, 20b) adsorption apparatus (30). Even if each layer (20a, 20b) of an adsorptive sintered compact (20) having such as columnar or prismatic or hollow shape is sequentially loaded or stacked in series, each layer (20a, 20b) does not mix with each other because they remain rigid. Although FIG. 5 shows a two-layer (20a, 20b) adsorption apparatus (30), it is also possible to form an adsorption apparatus stacked into three or more layers.

The following explains an embodiment of a method for producing an adsorptive sintered compact (20) according to the present disclosure using powdered activated carbon as a raw material.

First, powdered activated carbon as a raw material (powder adsorbent raw material) for constituting a powder adsorbent materials (1a, 1b) mixes with thermoplastic resin as a raw material of resin for constituting a resin structure (2) to form an adsorbent material mixture. An adsorbent material mixture may include other optional components, if necessary. The content of powdered activated carbon may be 25 to 65 weight % in the adsorbent material mixture, and may be 30 to 60 weight %. The content of thermoplastic resin may be 35 to 75 weight % in the adsorbent material mixture, and may be 40 to 70 weight %. A mean diameter of the powdered activated carbon may be less than 150 μm, and may be 1 μm or more and less than 150 μm, and may be 5 μm or more and less than 150 μm, and may be 15 μm or more and 100 μm or less. The particle diameter of the thermoplastic resin may be 10 to 200 μm, and may be 30 to 80 μm. Powdered activated carbon hardly crushes, expands, or shrinks, and can form the powder adsorbent materials (1a, 1b) while maintaining its original size. It can also be seen from, for example, FIGS. 2a and 4a, and FIGS. 2b and 4b showing images of the same magnification (400×) that the powder diameter of the powder adsorbent raw material is substantially equal to that of the powder adsorbent materials (1a, 1b) after production. The thermoplastic resin is preferably at least one selected from among polypropylene, polyethylene, polyvinylidene fluoride, and an ethylene-vinyl acetate copolymer. In the present disclosure, it is not necessary to use a foaming agent. The water content of the powdered activated carbon may be 30 weight % or less, may be 15 weight % or less, may be 8 weight % or less, and may be substantially free of water. If the water content of powdered activated carbon is low, in a heating step mentioned below, the adsorptive sintered compact can be stably formed for a shorter time while suppressing energy consumption without lowering the temperature in the vicinity of the powdered activated carbon. Incidentally, as to using at least one kind of thermoplastic resin from among polypropylene (PP), polyethylene (PE), polyvinylidene fluoride (PVDF), and an ethylene-vinyl acetate (EVA) copolymer preferably used in the present disclosure, these generally have poor water absorbency so it may not be necessary to consider the influence of water content in the method for producing an adsorptive sintered compact (20) of the present disclosure.

The water content of the powder adsorbent material may be measured as follows. 1 g to 3 g (w1) of powder adsorbent material is weighed and dried at 110° C. for a sufficient time until the mass change rate becomes 0.05%/min or less, and then the mass (w2) after drying is measured, with the water content (%) being 100×(w1−w2)/w2.

Next, the adsorbent material mixture is introduced into a heating furnace, and the adsorbent material mixture is heated at a temperature higher than the softening point of the thermoplastic resin and lower than the melting point of the powdered activated carbon, for example, at 90 to 180° C. By heating, the contact portions of a plurality of thermoplastic resins are melted to form joint portions, and the thermoplastic resins are fused to each other to form a skeleton for surrounding voids (3). In this case, most of the powdered activated carbon does not bond to the thermoplastic resin. Thereafter, it is cooled and solidified to form a three-dimensional network-shaped resin structure (2) in which free adsorbent materials (1a) are free-movably contained in voids (3). The resin structure (2) may have a cubic lattice-shaped three-dimensional network structure. This structure constitutes a high-strength adsorptive sintered compact (20) containing free adsorbent materials (1a) of powdered activated carbon. FIGS. 4a to 4c respectively illustrate embodiments of adsorptive sintered compacts (20) produced by the method of the present disclosure using powdered activated carbon as a raw material. In the production of an adsorptive sintered compact (20) of the present disclosure, by using powdered activated carbon and thermoplastic resin as resin raw materials preferably having lower water contents, it is possible to suppress excessive void expansion due to vigorous evaporation and foaming of water, and voids (3) having appropriate sizes can be uniformly formed. By using raw material having low water content, in the heating step, heat is uniformly transferred from the outside near a heating source to the inside without being affected by moisture, so that there is no temperature difference and the entire thermoplastic resins can be uniformly melted. In addition, almost no water is evaporated and released from the powdered activated carbon, and no external force is applied to the thermoplastic resin at the stage of forming the resin structures (2) so that there is no deforming, and thus voids (3) can be formed and maintained to a substantially uniform size within a predetermined range without expansion. Furthermore, from the viewpoint of uniformly forming such an appropriate dimension and voids (3), the particle diameter of the thermoplastic resin may be 10 to 200 μm, and may be 30 to 80 μm.

In the above-mentioned embodiment of a production method, powdered activated carbon is used as a raw material, but even if powdered activated clay or zeolite is used as a raw material, an adsorptive sintered compact (20) can be produced by the same production method as mentioned above (FIG. 4d and FIG. 4e).

An embodiment in which fluid (7), as liquid or gas, is passed through an adsorptive sintered compact (20) of the present disclosure, will be explained below by referring to FIG. 6.

When fluid (7) containing substance(s) to be treated is passed through an adsorption apparatus (not shown in the drawing) stacked as a single-layer adsorptive sintered compact (20), fluid (7) is introduced into voids (3) passing through gap (4) between resin structures (2). At this time, free adsorbent material (1a) is in a non-bonding state to resin structure (2), and a plurality of free adsorbent materials (1a) are not fixed or bonded to each other, so that, as shown in FIG. 6, free adsorbent materials (1a) are floated or swung in voids (3) by flowing fluid (7), and the entire surface of free adsorbent material (1a) comes into direct contact with fluid (7) and it can certainly capture and adsorb the substance. Additionally, by the flow path (3a) being formed between free adsorbent materials (1a), the adsorption area is increased, and flow of fluid (7) can be ensured and thus an increase in pressure loss can be prevented in an adsorptive sintered compact (20).

Also, a substance to be treated in the fluid (7) is captured and adsorbed by contacting the fluid (7) with the surface (2a) of the resin structure (2) or the fixed adsorbent material (1b) fixed inside thereof. Fluid (7) treated in the voids (3) passes through the gap (4) and is continuously introduced into other voids (3). The same adsorption treatment is repeated in the plurality of other voids (3), and finally the fluid (7) flows out to the outside of resin structure (2), that is, to the outside of the adsorption apparatus. Since the gap (4) is small, freely moving free adsorbent material (1a) is retained in the voids (3) between the resin structures (2) and does not flow out to the outside of the adsorptive sintered compact (20).

An adsorptive sintered compact (20) of the present disclosure can contain a high content of powder adsorbent material which is excellent in adsorption performance, and, therefore, it can perform an adsorption treatment of substance(s) with a high efficiency. When filling in a vessel such as a cartridge for use as a filter, the adsorption apparatus, such as the filter, can be miniaturized due to the small size of the vessel, and the transportability and storability can be made extremely good because an adsorptive sintered compact (20) of the present disclosure is also excellent in strength.

In an adsorptive sintered compact (20) of the present disclosure, a reduction in the adsorption performance due to an increase in pressure loss can be suppressed, and the adsorption performance can be maintained for a long period of time while retaining a predetermined treatment amount, so that reduction of running costs can be accomplished.

In adsorptive sintered compact (20) of the present disclosure, powdered activated carbon excellent in adsorption performance can be integrally molded with a desired shape and size as a sintered compact, and a continuous adsorption treatment with various forms becomes possible. Further, multiple functional adsorption apparatus can be provided because it is also possible to load the plurality of layers having different adsorption characteristics in vessel.

In addition, an adsorptive sintered compact (20) of the present disclosure can be used for a batch type adsorption treatment. In a batch type adsorption treatment using a powder adsorbent material, the powder adsorbent material is easily scattered in the air, i.e., it has a poor handling property, and there a local exhaust equipment may be required for preventing the deterioration of the working environment due to this powder dust; however, the adsorptive sintered compact (20) of the present disclosure also has an advantage in handling property.

### EXAMPLES

Examples of adsorptive sintered compact (20) according to the present disclosure will be explained as follows.

[1] Pressure Loss Test

With regard to pressure loss of an adsorptive sintered compact (20) (Examples 1 to 4) of the present disclosure, the following experiment was carried out with powder objects (69) (Comparisons 1 to 4 and Comparisons 1′ and 2′).

[1-1] Production of Adsorptive Sintered Compact (20) and Powder Object (69)

As raw materials of powder adsorbent materials (1a, 1b), 0.109 weight part of powdered activated carbon (AC)(FIG. 2a) having about 30 μm mean diameter, obtained by activating coconut shell carbide with water vapor, and 0.164 weight part of Polyethylene (PE) powder as resin raw material of resin structure (2) were mixed, and heated and sintered at about 125° C. to obtain an adsorptive sintered compact (20) (FIG. 4a) (Example 1) of the present disclosure containing 40 weight % of powder adsorbent materials (1a, 1b). Similarly, as raw materials of powder adsorbent materials (1a, 1b), 0.109 weight part of powdered AC (FIG. 2b) having about 42 μm mean diameter, obtained by activating sawdust with chemical (phosphoric acid), and 0.109 weight part of PE powder as resin raw material of resin structure (2) were mixed, and heated and sintered at about 125° C. to obtain an adsorptive sintered compact (20) (FIG. 4b) (Example 2) of the present disclosure containing 40 weight % of powder adsorbent materials (1a, 1b). The water content of the powdered ACs as raw material used in Examples 1 and 2 were each about 7 weight %. As raw materials of powder adsorbent materials (1a, 1b), 0.139 weight part of powdered activated clay (FIG. 2d) having about 11 μm mean diameter and 0.139 weight part of PE powder as resin raw material of resin structure (2) were mixed, and heated and sintered at about 125° C. to obtain an adsorptive sintered compact (20) (FIG. 4d) (Example 3) of the present disclosure containing 50 weight % of powder adsorbent materials (1a, 1b). As raw materials of powder adsorbent materials (1a, 1b), 0.139 weight part of zeolite (FIG. 2e) having about 35 μm mean diameter and 0.139 weight part of PE powder as resin raw material of resin structure (2) were mixed, and heated and sintered at about 125° C. to obtain an adsorptive sintered compact (20) (FIG. 4d) (Example 4) of the present disclosure containing 50 weight % of powder adsorbent materials (1a, 1b). The water content of the powdered activated clay and zeolite as raw materials used in Examples 3 and 4 were each about 7 weight %.

0.109 weight part of powdered AC (FIG. 2a) having about 30 μm mean diameter, obtained by activating coconut shell carbide with water vapor, was produced as a powder object (69) (Comparison 1). 32 μm or less of powdered AC was classified and removed by vibration classifier (manufactured by Fritsch Co., Ltd.) from powdered AC (FIG. 2a) having about 30 μm mean diameter, obtained by activating coconut shell carbide with water vapor, to obtain 0.109 weight part of powdered AC as a powder object (69) (Comparison 1′). 0.109 weight part of powdered AC (FIG. 2b) having about 42 μm mean diameter, obtained by activating sawdust with chemical (phosphoric acid), was produced as a powder object (69) (Comparison 2). 32 μm or less of powdered AC was classified and removed by vibration classifier from powdered AC (FIG. 2b) having about 42 μm mean diameter, obtained by activating sawdust with chemical (phosphoric acid), to obtain 0.109 weight part of powdered AC as powder object (69) (Comparison 2′). 0.109 weight part of powdered activated clay (FIG. 2d) having about 11 μm mean diameter was produced as a powder object (69) (Comparison 3). 0.109 weight part of zeolite (FIG. 2e) having about 35 μm mean diameter was produced as a powder object (69) (Comparison 4).

[1-2] Test Method

### Examples 1 to 4

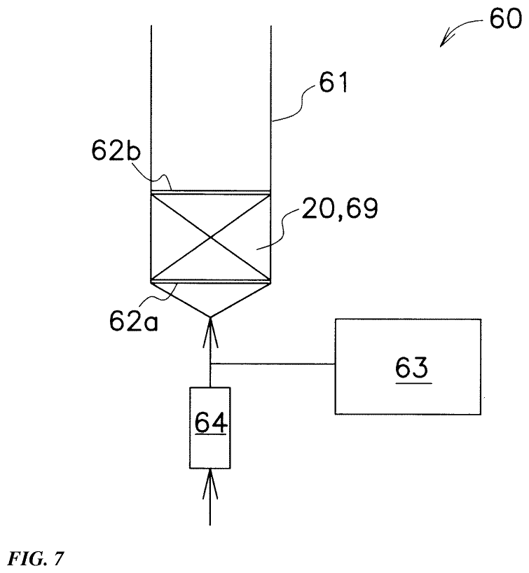

A frit (filter for preventing powder leakage) (62a) was arranged in each syringe (61) having a volume of about 3 ml, each adsorptive sintered compact (20) of Examples 1 to 4 was loaded thereon, and further frit (62b) was arranged to make a pressure loss test apparatus (60) in FIG. 7. The pressure difference of the adsorptive sintered compact (20), when air adjusted to 0.2 L/min by a flow meter (64), was passed through the adsorptive sintered compact (20), was measured by a pressure gauge (AP-53A manufactured by KEYENCE CORPORATION) (63) with regard to each of Examples 1 to 4.

<Comparisons 1 to 4 and Comparisons 1′ and 2′>

In the same test method as the above Examples, except that syringes (61) were filled with each of the powder objects (69) of Comparisons 1 to 4 and Comparisons 1′ and 2′ in place of adsorptive sintered compact (20), the differential pressure of each Comparison was measured.

[1-3] Test Results and Consideration

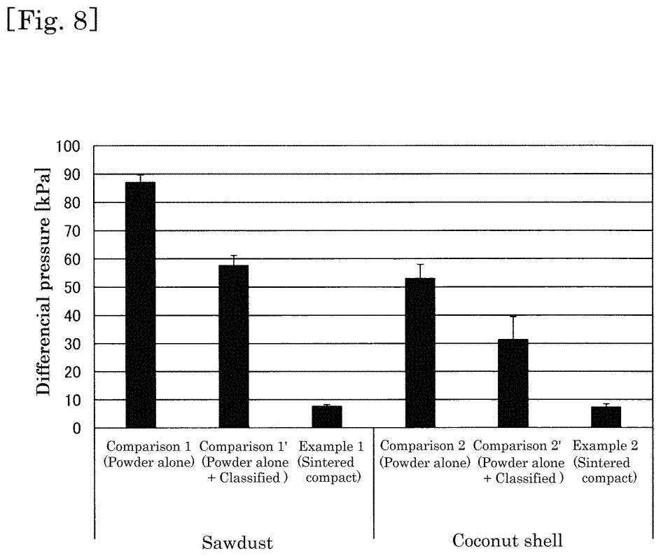

As shown in the pressure loss test results of FIG. 8, in which the name of test object is shown on the horizontal axis and the differential pressure [kPa] is shown on the vertical axis, Comparisons 1 and 2 filled in with powdered AC showed a high differential pressure value of 50 kPa or more, and Comparisons 1′ and 2′, in which powdered AC of 32 μm or less was classified and removed, also showed high differential pressure value of 30 kPa or more. Comparisons 1 and 2 were filled with powdered AC containing a fine powder of 32 μm or less with a high density, and Comparisons 1′ and 2′ were also filled with a powdered AC with a high density, so that the space between powders is small and thus these showed high pressure loss. To the contrary, adsorptive sintered compacts (20) according to the present disclosure of Examples 1 and 2 each showed low differential pressure value of 10 kPa or less in spite of containing a resin structure (2) in addition to the same amount of powdered AC as Comparisons. Comparisons 3 and 4, in which a powder object (69) of powdered activated clay and zeolite was filled, showed high differential pressure value of 29 kPa and 97 kPa, respectively. Comparisons 3 and 4 showed a high pressure loss since fine powder was filled at a high density and the space between powders was small. To the contrary, an adsorptive sintered compacts (20) according to the present disclosure of Examples 3 and 4 each showed a low differential pressure value of 10 kPa or less in spite of containing resin structure (2) in addition to the same amount of powdered activated clay and zeolite as Comparisons 3 and 4. Accordingly, in Examples 1 to 4 of the present disclosure, it was confirmed that a lower pressure loss treatment was realized. Further, the adsorptive sintered compacts (20) of Examples 1 to 4 were also excellent in strength.

[2] Liquid Flow Adsorption Test

With regard to the liquid flow adsorption performance of adsorptive sintered compacts (20) (Examples 5 and 6) of the present disclosure, the following test was carried out together with granular objects (89) (Comparisons 5 and 6) using granular activated carbon.

[2-1] Production of Adsorptive Sintered Compact (20) and Granular Object (89)

As raw materials of powder adsorbent materials (1a, 1b), 0.076 weight part of powdered AC (FIG. 2a) having about 30 μm mean diameter, obtained by activating coconut shell carbide, and 0.177 weight part of PE powder as resin raw material of resin structure (2) were mixed, and heated and sintered at about 125° C. to obtain a high strength adsorptive sintered compact (20) (FIG. 4a) (Examples 5) of the present disclosure containing 30 weight % of powder adsorbent materials (1a, 1b). Similarly, as raw materials for constituting powder adsorbent materials (1a, 1b), 0.076 weight part of powdered AC (FIG. 2b) having about 42 μm mean diameter, obtained by activating sawdust with a chemical (phosphoric acid), and 0.177 weight part of PE powder as resin raw material of the resin structure (2) were mixed, and heated and sintered at about 125° C. to obtain a high strength adsorptive sintered compact (20) (FIG. 4b) (Examples 6) of the present disclosure containing 30 weight % of powder adsorbent material (1a, 1b). The water content of powdered AC used in Examples 5 and 6 was about 8 weight %.

0.076 weight part of granular activated carbon having about 800 μm mean diameter, obtained by activating coconut shell carbide with water vapor, and 0.177 weight part of PE beads having 100 μm diameter were mixed to obtain a granular object (89) (Comparison 5). In the large diameter granular activated carbon having about 800 μm diameter, an adsorptive sintered compact of the present disclosure in which AC was free-movably arranged in voids between resin structures could not be formed, and the number of grains was extremely small as compared with that of powder so that voids without AC were formed with a large number, so that the same amount of PE beads as that of PE powder was used in order to meet the same conditions as in Example 5 as much as possible. Similarly, 0.076 weight part of granular activated carbon having about 800 μm mean diameter, obtained by activating sawdust raw material with a chemical (phosphoric acid), and 0.177 weight part of PE beads having 100 μm diameter were mixed to obtain a granular object (89) (Comparison 6). Incidentally, 800 μm mean diameter corresponds to the particle diameter of a granular activated carbon frequently used in a liquid phase treatment.

[2-2] Test Method

### Examples 5 and 6

Using a liquid flow adsorption test apparatus (80), 20 ml of methylene blue solution having a concentration of 1,200 mg/l stored in a container (82) was fed by a tube pump (83) at 3.2 ml/min to a vessel (81) having an inner diameter of 8.6 mm loaded with each of the adsorptive sintered compacts (20) of Examples 5 and 6 and was circulated (FIG. 10). After collecting every 0.1 ml of the circulating fluid at constant intervals and diluting them 100 times, the absorbance at wavelength of 665 nm was measured by a spectrophotometer (UV-1700 manufactured by Shimadzu Corporation), and the residual concentration of methylene blue in the circulating fluid was obtained for Examples 5 and 6.

<Comparisons 5 and 6>

In the same test method as the above Examples 5 and 6, except that the granular objects (89) of Comparisons 5 and 6 filled therewith in each vessel (81) were used in place of the adsorptive sintered compact (20), the residual concentration of methylene blue was obtained for Comparisons 5 and 6.

[2-3] Test Results and Consideration

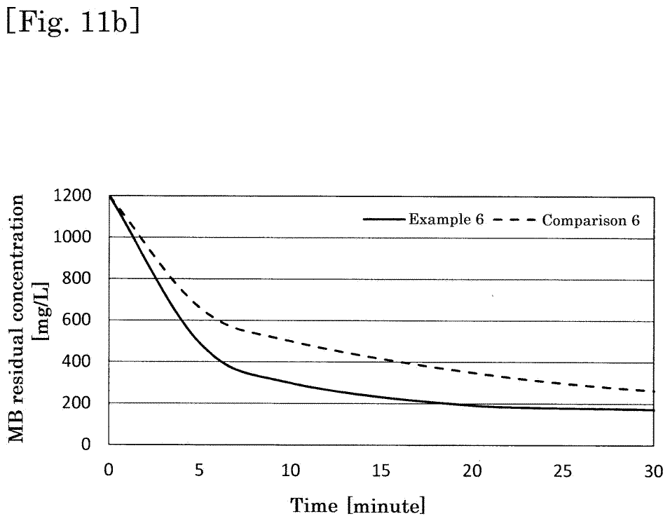

FIG. 11a shows test results of Example 5 and Comparison 5, and FIG. 11b shows test results of Example 6 and Comparison 6, and time [min] is shown on the horizontal axis, and residual concentration [mg/l] of methylene blue is shown on the vertical axis, respectively. For example, when the residual concentrations of methylene blue after 10 min were compared to each other, from FIG. 11a, Comparison 5 was about 780 mg/l, and Example 5 decreased to about 300 mg/l. Also, from FIG. 11b, Comparison 6 was about 500 mg/l, and Example 6 decreased to 300 mg/l. Therefore, as compared with Comparisons 5 and 6 containing granular activated carbon alone having 800 μm diameter, Examples 5 and 6 of the present disclosure containing 30 weight % of powder adsorbent materials (1a, 1b) could be confirmed that the adsorption performance of liquid, particularly, the decoloration property was excellent. Also, the adsorptive sintered compacts (20) of Examples 5 and 6 were excellent also in strength.

[3] Gas Flow Adsorption Test

With regard to the gas flow adsorption performance of an adsorptive sintered compact (20) (Example 5) of the present disclosure, the following test was carried out together with granular objects (99) (Comparisons 7 and 8) using granular activated carbon.

[3-1] Production of Adsorptive Sintered Compact (20) and Granular Object (99)

An Adsorptive Sintered Compact (20) of Example 5 was Obtained by the same method as mentioned above. 0.076 weight part of granular activated carbon having about 2,000 μm mean diameter, obtained by activating coconut shell carbide with water vapor, and 0.177 weight part of PE beads having 100 μm diameter were mixed to obtain a granular object (99) (Comparison 7). In the large diameter granular activated carbon having about 2,000 μm particle diameter, an adsorptive sintered compact of the present disclosure in which AC was free-movably arranged in voids between resin structures cannot be formed, and the number of grains was extremely small as compared with that of powder so that voids without activated carbon were formed in a large number, so that the same amount of PE beads as that of PE powder was used in order to meet the same conditions as in Example 5 as much as possible. A granular object (99) (Comparison 8) containing only 0.076 weight part of granular activated carbon having about 2,000 μm mean diameter was obtained by activating coconut shell carbide with water vapor. Incidentally, 2,000 μm mean diameter corresponds to the particle diameter of a granular activated carbon frequently used in gas phase treatment.

[3-2] Test Method

### Example 5

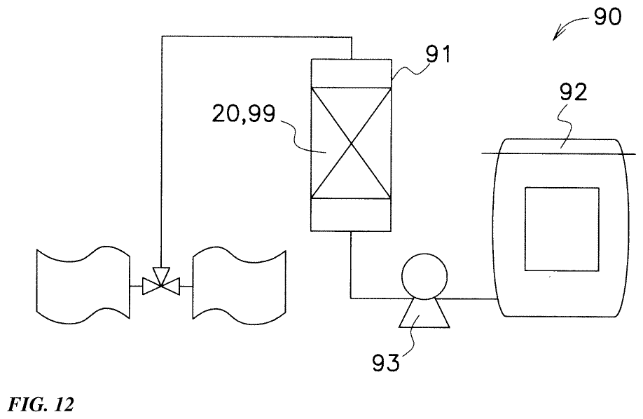

Using a gas flow adsorption test apparatus (90) shown in FIG. 12, cyclohexane gas was supplied at 0.2 ml/min by a diaphragm pump (93), from a gas capturing bag (92) adjusted to about 100 ppm of cyclohexane gas by arranging therein a cyclohexane impregnated source, to a vessel (91) having an inner diameter of 8.6 mm loaded with an adsorptive sintered compact (20) (Example 5). The outlet gas, after passing through the adsorptive sintered compact (20), was collected in an amount of 2 L in 10 minutes, and the cyclohexane concentration every 10 minutes was measured by a gas detector tube (GASTEC CORPORATION 102L). In addition, the odor at the outlet gas was evaluated by a sensory test.

<Comparisons 7 and 8>

Using the same test method as the above Example 5, except that granular objects (99) of Comparisons 7 and 8 filled therewith in each vessel (81) were used in place of an adsorptive sintered compact (20), the cyclohexane concentration was measured, and the sensory test was carried out.

[3-3] Test Results and Consideration

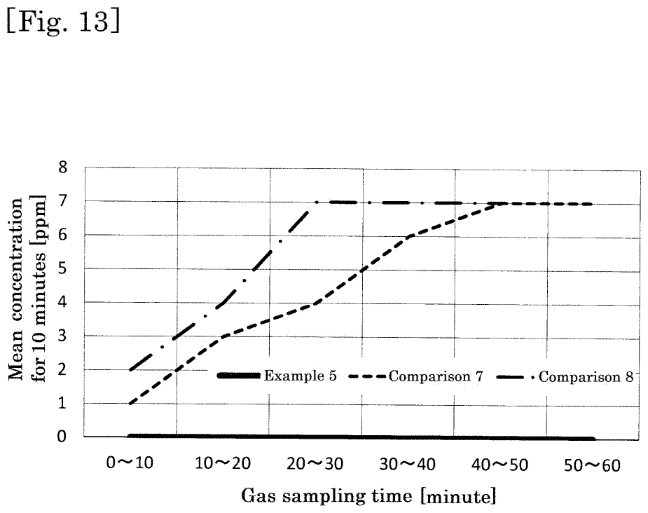

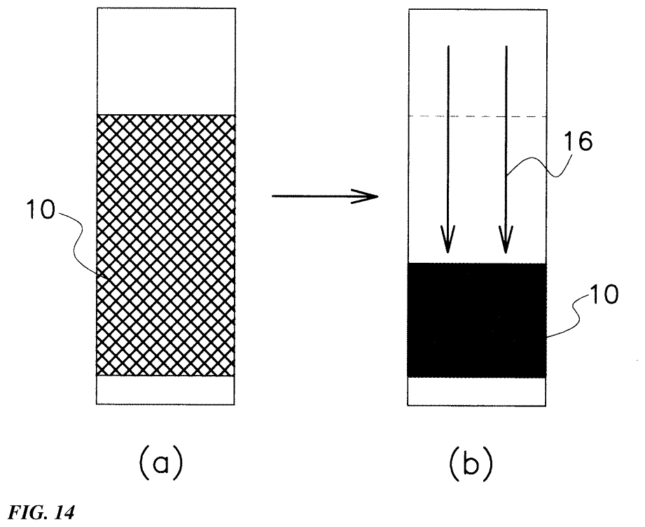

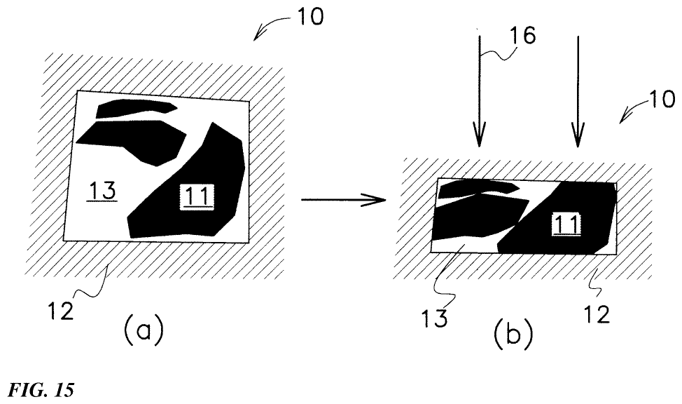

FIG. 13 shows the test results of Example 5 and Comparisons 7 and 8, and time [min] is shown on the horizontal axis, and cyclohexane concentration [ppm] is shown on the vertical axis. In Comparisons 7 and 8, cyclohexane broke through from the first 10 minutes, whereas in Example 5, cyclohexane did not break through even after 60 minutes, and the adsorption was completely maintained. In addition, as a result of the sensory test, in Comparisons 7 and 8, a cyclohexane odor of the outlet gas was detected from the first 10 minutes, whereas in Example 5, no odor was detected. Therefore, Example 5 of the present disclosure containing 30 weight % of powder adsorbent materials (1a, 1b) could be confirmed that it was excellent in the adsorption performance of gas, particularly, a deodorizing property and toxic gas removing characteristics.

[4] Sinterability and Strength Test

Sinterability and the strength of an adsorptive sintered compact (20) containing powder adsorbent materials (1a, 1b) were confirmed.

[4-1] Manufacture and Test Method of an Adsorptive Sintered Compact (20)

Powdered AC (FIG. 2b) having about 42 μm mean diameter, obtained by activating sawdust with a chemical (phosphoric acid), as the raw material for constituting powder adsorbent materials (1a, 1b) and PE powder for forming resin structure (2) were mixed so that an adsorptive sintered compact (20) became 0.273 g, and heated and sintered at about 125° C., whereby adsorptive sintered compacts (20) (Examples 7 and 8) containing 50 and 60 weight % of powder adsorbent materials (1a, 1b) were obtained, respectively. The reason why powdered AC from sawdust is used is that, in the above Examples 1 to 6, sawdust has relatively poor strength as compared with the other raw materials, and if high sinterability and high strength can be confirmed in sawdust powdered AC, it is naturally predictable that sufficient sinterability and strength can be also obtained from the other coconut shell powdered AC, powdered activated clay, and zeolite.

[4-2] Test Method

With regard to Examples 7 to 8, sinterability was confirmed by visual inspection of appearance and strength by touching hands, respectively.

[4-3] Test Results and Consideration

Adsorptive sintered compacts (20) containing 30 weight % (Example 6), 40 weight % (Example 2), 50 weight % (Example 7) and 60 weight % (Example 8) of powder adsorbent materials (1a, 1b) each could be taken out from a heating furnace in a state maintaining its shape, and the shape did not change even when pressed strongly, so that sufficient strength could be confirmed.

[5] Batch Type Liquid Phase Adsorption Test

With regard to the batch type liquid phase adsorption performance of an adsorptive sintered compact (20) of the present disclosure, the following test was carried out together with powdered activated clay (FIG. 2d).

[5-1] Manufacture of Adsorptive Sintered Compact (20)

As the raw materials of powder adsorbent materials (1a, 1b), 0.197 weight part of powdered activated clay (FIG. 2d) having about 11 μm mean diameter and 0.131 weight part of PE powder as resin raw material of resin structure (2) were mixed, and heated and sintered at about 125° C. to obtain an adsorptive sintered compact (20) of the present disclosure (Examples 9) containing 60 weight % of powder adsorbent materials (1a, 1b). The water content in the powdered activated clay was about 7 weight %.

[5-2] Test Method

### Example 9

Into an 100 ml Erlenmeyer flask was charged a adsorptive sintered compact (20) of Example 9, and 50 ml of caffeine aqueous solution having concentration of 100 mg/l was added thereto, and the flask was closed with a rubber stopper and set on a shaker and shaken at normal temperature (20° C.) at 200 rpm until adsorption reaches almost equilibrium. The liquid after shaking was collected, caffeine was separated using a high performance liquid chromatograph device (Chromaster (Registered Trademark) manufactured by Hitachi High-Tech Science Corporation), and the residual concentration of caffeine for Example 9 was measured from absorbance at wavelength of 280 nm to obtain the removal rate of caffeine by adsorption.

<Comparison 9>

Using the same test method as the above Example 9, except that 0.197 weight part of powdered activated clay (FIG. 2d) was used in place of the adsorptive sintered compact (20), the removal rate of caffeine by adsorption was obtained.

[4-3] Test Results and Consideration

Both Example 9 and Comparison 9 showed an equal value of 97% in the removal rate of caffeine by adsorption. Therefore, it could be confirmed that adsorption performance of powder adsorbent materials (1a, 1b) was highly maintained even in an adsorptive sintered compact (20). In addition, an adsorptive sintered compact (20) of Example 9 was also excellent in strength.

[6] Batch Type Gas Phase Adsorption Test

With regard to the batch type gas phase adsorption performance of an adsorptive sintered compact (20) of the present disclosure, the following test was carried out together with chemical-supported zeolite.

[6-1] Manufacture of Adsorptive Sintered Compact (20)

As the raw materials of powder adsorbent materials (1a, 1b), 0.055 weight part of chemical-supported zeolite, pulverized to about 35 μm mean diameter, and 0.055 weight part of PE powder as the resin raw material of a resin structure (2) were mixed, and heated and sintered at about 125° C. to obtain a high strength adsorptive sintered compact (20) (Example 10) of the present disclosure containing 50 weight % of powder adsorbent materials (1a, 1b). The water content of chemical-supported zeolite was about 7 weight %.

[6-2] Test Method

### Example 10

Into a 3 L odor bag (manufactured by Omi Odor Air Service Co., Ltd.) made of polyester were placed an adsorptive sintered compact (20) of Example 10 and a filter paper piece of about 3 cm square, and 3 L of clean air was added therein and the bag was sealed with a rubber stopper. 3% dimethyl sulfide (hereinafter referred to as DMS) solution was injected into the odor bag with a micro syringe so that internal DMS concentration became 200 mg/m³ to impregnate it into the filter paper piece, and the injection port was sealed with cellophane tape. DMS was vaporized inside the odor bag, and the bag was left at rest at normal temperature (20° C.) until adsorption was almost at equilibrium. The rubber stopper was opened and the DMS concentration in the odor bag was measured using a gas detecting tube No. 77 manufactured by GASTEC CORPORATION to obtain the removal rate of DMS by adsorption.

<Comparison 10>

Using the same test method as the above Example 10, except that 0.055 weight part of chemical-supported zeolite pulverized to about 35 μm mean diameter was used in the place of the adsorptive sintered compact (20), the removal rate of DMS by adsorption was obtained.

[6-3] Test Results and Consideration

The removal rate of DMS by adsorption of Examples 10 was 96%, and removal rate of DMS by adsorption of Comparison 10 was 97%, which were almost the same values. Therefore, it could be confirmed that the adsorption performance of powder adsorbent materials (1a, 1b) was highly maintained even when in an adsorptive sintered compact (20). In addition, the adsorptive sintered compact (20) of Example 10 was also excellent in strength.

[7] Conclusion of Examples

From the above Examples, it could be confirmed that an adsorptive sintered compact (20) of the present disclosure had high strength and exhibited excellent adsorption performance, such as decoloration and deodorization, even at low pressure loss in liquid and gas.

### UTILIZABILITY IN INDUSTRY

An adsorptive sintered compact, a production method therefor, and an adsorption apparatus of the present disclosure can be used for various uses such as air purification, dioxin removal, flue gas desulfurization and denitrification, waste gas and liquid treatment of factory, advanced water purification, purification of chemicals, decolorization of foods and beverages, water purifiers for home use, air purifiers, refrigerator deodorants, gas masks, etc.

### EXPLANATION OF REFERENCE NUMERALS

(1a, 1b) . . . Powder adsorbent material
(1a) . . . Tree adsorbent material
(1b) . . . Fixed adsorbent material
(2) . . . Resin structure
(2a) . . . Surface
(2b) . . . Concave portion
(3) . . . Voids
(20) . . . Adsorptive sintered compact
(30) . . . Adsorption apparatus

We claim:

1. An adsorptive sintered compact comprising:
powder adsorbent materials; and
a resin structure including voids formed in a three-dimensional network;
wherein the powder adsorbent materials include
a free adsorbent material free-movably contained in the voids between the resin structures, and
a fixed adsorbent material fixed to a surface of the resin structure and/or at least partly embedded inside the resin structure; and
wherein the powder adsorbent materials comprise at least one of powdered activated carbon, powdered activated clay, and zeolite.

2. The adsorptive sintered compact according to claim 1, wherein a mean diameter of the powder adsorbent materials is less than 150 μm.

3. The adsorptive sintered compact according to claim 1, wherein the powder adsorbent materials are contained in an amount of 25 to 65 weight % of said compact.

4. The adsorptive sintered compact according to claim 1, wherein:
said resin structure is formed from a resin raw material; and
the resin raw material of the resin structure comprises at least one thermoplastic resin selected from the group consisting of polypropylene (PP), polyethylene (PE), polyvinylidene fluoride (PVDF), and ethylene-vinyl acetate (EVA) copolymer.

5. The adsorptive sintered compact according to claim 4, wherein a particle diameter of the at least one thermoplastic resin is 10 to 200 μm.

6. The adsorptive sintered compact according to claim 1, wherein:
a plurality of free adsorbent materials are free-movably contained in at least a part of the voids, and
the plurality of the adjacent free adsorbent materials form a flow path for fluid between free adsorbent materials in at least a part of the voids without fixation of the adjacent free adsorbent materials to each other.

7. The adsorptive sintered compact according to claim 1, wherein the powder adsorbent material is powdered activated carbon having a sharp portion.

8. The adsorptive sintered compact according to claim 1, configured to adsorb a substance to be treated in a fluid.

9. An adsorption apparatus comprising:
a vessel; and
at least one layer of the adsorptive sintered compact according to claim 1 loaded in the vessel.

10. A method for producing an adsorptive sintered compact, the method comprising:
mixing at least one powder adsorbent material selected from the group consisting of powdered activated carbon, powdered activated clay, and zeolite, with a thermoplastic resin to form an adsorbent material mixture;
heating the adsorbent material mixture at a temperature higher than a softening point of the thermoplastic resin and lower than a melting point of a raw material of the at least one powder adsorbent material; and
forming a resin structure in which a plurality of the thermoplastic resins are fused and solidified by cooling to form voids in a three-dimensional network and free adsorbent material free-movably contained in the voids.

11. The method according to claim 10, wherein a mean diameter of the powder adsorbent material is less than 150 μm.

12. The method according to claim 10, wherein a content of the powder adsorbent material is 25 to 65 weight % in the adsorbent material mixture.

13. The method according to claim 10, wherein the thermoplastic resin comprises at least one resin selected from the group consisting of polypropylene (PP), polyethylene (PE), polyvinylidene fluoride (PVDF), and ethylene-vinyl acetate (EVA) copolymer.

14. The method according to claim 10, wherein a particle diameter of the thermoplastic resin is 10 to 200 μm.
